(19) United States
(12) Patent Application Publication
(10) Pub. No.: US 2021/0245339 A1
(43) Pub. Date: Aug. 12, 2021

(54) Torque-Limiting Device And Method For The Calibration Thereof

(71) Applicant: Wera Werkzeuge GmbH, Wuppertal (DE)

(72) Inventor: Michael Abel, Radevormwald (DE)

(73) Assignee: Wera Werkzeuge GmbH, Wuppertal (DE)

(21) Appl. No.: 17/251,102

(22) PCT Filed: Jun. 14, 2019
(86) PCT No.: PCT/EP2019/065691

§ 371 Date: Dec. 10, 2020

(30) Foreign Application Priority Data
Jun. 15, 2018 (DE) 10 2018 114 431.8

(51) Int. Cl. B25B 23/142; A61B 17/88; B25B 23/14; B25B 23/00

(52) U.S. Cl. CPC B25B 23/1427; A61B 17/8875; B25B 15/005; B25B 23/0042; B25B 23/141

(57) ABSTRACT

A torque-limiting device having two torque transfer bodies, which, as a consequence of oblique flank contact during relative rotation about an axis, can be moved relative to each other in the direction of the axis against the restoring force of a spring element, which is supported on a shoulder of an abutment, the position of which shoulder is set during calibration of a limiting torque. The torque-limiting device is calibrated by applying a predefined force to the spring element. The resulting spring length defines the axial position of the shoulder of the abutment, which axial position is irreversibly fixed.

### TECHNICAL FIELD

The present invention relates to a torque-limiting device having two torque transfer bodies, which, as a consequence of an oblique flank contact during relative rotation about an axis, can be moved relative to each other in the direction of said axis against the restoring force of a spring element, which is supported on a shoulder of an abutment, the position of which shoulder is set during calibration of a limiting torque. Moreover, the invention relates to a calibration method for calibrating a torque transfer device having two torque transfer bodies, which, as a consequence of an oblique flank contact during relative rotation about an axis, can be moved relative to each other in the direction of said axis against the restoring force of a spring element, which is supported on a shoulder of an abutment, the position of which shoulder is set during calibration of a limiting torque. Moreover, the invention relates to a plurality of screwdrivers, which each comprise an identically designed torque-limiting device.

### BACKGROUND

A generic torque-limiting device is described in DE 10 2014 212 222 A1. A shaft produced from steel comprises at its free end a screwdriver profile. The opposite end of the shaft has an outer thread, on which a nut is screwed. A first torque transfer body, which is connected rotationally fixed and axially fixed to the shaft, is supported on a projection of the shaft. The torque transfer body has torque transfer teeth with tooth flanks arranged saw tooth-like. There is supported on an obliquely running tooth flank of a torque transfer tooth an obliquely running tooth flank of a second torque transfer body, on which a spring element acts in the axial direction of the shaft. The spring element is supported on the nut screwed onto the outer thread of the shaft. This torque-limiting device can be incorporated in a screwdriver, for example the torque transfer device, as shown in EP 1 631 418 B1, can be inserted into a handle cavity of a screwdriver handle. The torque-limiting device can be calibrated by means of the nut. By rotating the nut, the pre-tensioning of the spring element changes with which the second torque transfer body is acted upon in the axial direction. If a torque is generated between the two torque transfer bodies, this leads to a relative rotation of the second torque transfer body, which is assigned to the shaft in an axially mobile and rotationally mobile manner, with respect to the first torque transfer body, wherein the two oblique flanks slide over one another during the relative rotation, with the consequence that the two torque transfer bodies move axially away from one another. In the course of this axial movement of the second torque transfer body, the spring element is tensioned until the torque transfer tooth of the second torque transfer body runs over the torque transfer tooth of the first torque transfer body. The spring pre-tensioning then reached corresponds to a release torque, which when reached no longer transmits any further torque from one torque transfer body to the other torque transfer body, because the two torque transfer bodies rotate relative to one another when this limiting torque is exceeded.

In the prior art, this release torque is set by rotating the nut.

In the prior art, moreover, there are torque-limiting devices which do not enable any calibration of the release torque. In the production of such torque-limiting devices, use is made of spring elements, the spring constant whereof lies within a narrow tolerance range. An initial calibration can be carried out here by the use of washers. Such a screwdriver is described in EP 1 092 510 B1.

DE 10 2016 013 174 A1 describes a very small torque wrench with a flat housing, out of which a shaft mounted in the housing projects. Two torque transfer bodies are mounted on the shaft, wherein one torque transfer body is applied against the other torque transfer body by a compression spring. The force with which the compression spring is applied against the torque transfer body is set during the assembly of the two housing parts. The two housing parts are applied against one another with a predefined force and welded together in this state.

### SUMMARY OF THE INVENTION

The problem underlying the invention is to specify a screwdriver which can be calibrated with straightforward means and to propose measures with which the calibrated release torque cannot be subsequently changed, and to specify a method with which such a calibration is possible.

The problem is solved by the invention specified in the claims, wherein the sub-claims not only represent advantageous developments of the coordinated claims, but also independent solutions to the problem.

In the first place and essentially, it is proposed that the calibration of the torque-limiting device does not take place by measuring a release torque or limiting torque that can be transferred from the torque transfer bodies, but that the spring element is pretensioned using a predefined force, wherein an axial position of the abutment is set by the pretensioning of the spring element, in which position the abutment is fixed irreversibly. Due to the fact that the abutment is irreversibly fixed, a release torque once set can no longer be changed. This increases the manipulation reliability of a torque tool calibrated to a specific torque value. The fixing of the abutment in the axial position found by applying a predefined force preferably takes place while the spring element is still acted upon by the predefined force. This can take place, for example, by the fact that the spring element or intermediate element lying on the spring element, such as a washer for example, is acted upon by to a predefined weight force. For this purpose, standard weights can be used. Since the spring elements, which are preferably constituted by helical compression springs, have lengths subject to tolerances and spring constants subject to tolerances, the pretensioned spring elements acquire an individual axial length, in which the preferably helical compression spring acted upon and compressed by the predefined force is fixed in position. The axial position of the abutment can be set irreversibly in different ways, for example by means of a form-fit connection, a friction-locked connection or a firmly bonded connection to a shaft or to another element of the torque-limiting device, which is connected axially fixed to the torque transfer body. For example, it is possible to connect an abutment body by means of a spot weld or a solder joint to a free end of a shaft. The shoulder can however also be constituted by a spot weld itself. It is also possible to connect an abutment body by means of an adhesive joint to the shaft. Form-fit connections of an abutment body to the shaft are also possible, for example by a creating a drill hole and inserting a splint, wherein the spatial assignment of the abutment body for example to the shaft of the torque-limiting device takes place with a pretensioned spring element. Instead of the splint, use can also be made of a wedge, which is introduced into a longitudinal slot of the shaft and is fixed there irreversibly. A nut known from the prior art can be irreversibly rotationally fixed for example by means of a spot weld or suchlike. In a preferred embodiment of the torque-limiting device according to the invention, the abutment is produced by a plastic deformation. In particular, provision is made such that a free end of the shaft is plastically deformed, so that a shoulder is formed, on which the spring element, preferably the helical compression spring produced from steel, is supported, optionally with the interposing of an intermediate element, for example a washer. The other end of the spring element can be supported on a torque transfer body axially mobile with respect to the shaft and rotatable with respect to the shaft, said torque transfer body forming oblique flanks which lie against oblique flanks of a further torque transfer body, which is connected axially and rotationally fixed to the shaft. A second intermediate element, also in the form of a washer, can be provided, which is assigned to the second torque transfer body. The spring element can thus be supported with both ends in each case on a washer. The first torque transfer body, which is connected rotationally fixed to the shaft, can be placed on an insert piece. The insert piece can plug into a cavity of the second torque transfer body. The insert piece is preferably a hexagonal driver plate, which has an outer hexagon and an inner hexagonal opening. The cavity of the torque transfer body has an inner hexagonal profile, in which the hexagonal outer profile of the hexagonal driver plate lies in a form-fit manner. The flanks of the inner hexagonal profile of the hexagonal driver plate abut against a hexagonal section of the shaft. The torques that can be transferred by the shaft to the second torque transfer body are increased with the hexagonal driver plate. In order to move the two torque transfer bodies relative to one another in the axial direction when a relative torque is applied, a lifting device can be provided. This lifting device can be constituted by torque transfer teeth, which form oblique flanks which lie against one another and slide over one another during the relative movement. Alternative embodiments of a lifting device are also possible. Thus, spheres can be provided which lie in spherical receiving openings, wherein each of the two torque transfer bodies comprises one or more spherical receiving openings.

In the method according to the invention, a plurality of essentially structurally identical torque-limiting devices can be calibrated in a straightforward manner. For this purpose, use is made in each case of torque transfer bodies which comprise torque transfer teeth, which have obliquely running tooth flanks which lie in a surface contact with one another in the state when no torque is applied. Provision can however also be made such that only one torque transfer body comprises one or more oblique flanks, against which for example a rounded portion or other marginal edge of the other torque transfer body abuts. It is essential that the gradient of the oblique flank of the at least one torque transfer body constituting an oblique flank lies in a narrow tolerance field. These production-related boundary conditions can be adequately implemented if the torque transfer bodies are plastic or metal injection moulded parts, sintered parts, milled parts or precision cast parts. The inclination and the axial distance of the oblique surface from the rotational axis defines the release torque with a predefined force.

In the calibration method according to the invention, use is made of spring elements which, on account of tolerances, have different lengths from one another and spring constants differing from one another. According to the invention, the calibration of the torque-limiting device takes place during its assembly, wherein the two torque transfer bodies are first mounted on a shaft, if need be a washer is placed on the uppermost torque transfer body and then the spring element, which can be a steel spring, is positioned on the uppermost torque transfer body. Optionally, an intermediate element, for example a washer, can be placed on the end of the spring element pointing upwards. A section of the shaft, on which the lower torque transfer body is supported, extends above the spring element or the intermediate element. In a calibration step, a predefined force, for example the weight force of a standard weight, is now applied to the spring element from above. The effect of this is that the spring element is compressed to an individual length. The spring element is then fixed in position in this calibrated length. This takes place by the positioning of a shoulder of an abutment, wherein provision is in particular made such that the shoulder is produced by a plastic deformation when the fixing in position of the upward pointing end of the spring element takes place. For example, two press jaws can be provided, which are applied to the free end of the shaft in the radial direction, so that it is reduced in cross-section and forms shoulders extending perpendicular to the force application, on which shoulders the spring element or the intermediate element can be supported. In this variant of the invention, a basic length of the spring element is set with the predefined force, which is further reduced with the application of a torque on the two torque transfer bodies up to a release length, at which the torque transfer tooth of the rotatable torque transfer body runs over the torque transfer tooth of the torque transfer body connected rotationally fixed to the shaft. In a variant of the invention, however, provision can also be made such that the spring pretensioning is set by the predefined force, said spring pretensioning corresponding to the release torque. In this second method variant, a larger predefined force is used than in the first method variant. Whereas in the first method variant the abutment is produced or fixed exactly at the position at which the spring element of the intermediate element is to be supported, the abutment in the case of the second method variant can be produced at an axial distance from the upper end of the spring element or of the intermediate element, wherein the distance between the upper end of the spring element or the upper end of the intermediate element from the abutment or the shoulder forming the abutment corresponds to the axial travel path of the mobile torque transfer body, i.e. for example the height of a torque transfer tooth. As an alternative to this, the two torque transfer bodies can also be brought into a rotational position during the position-fixing of the abutment, in which rotational position the torque transfer teeth rest against one another, the torque transfer bodies thus acquire a spacing position which is reached when the release torque is exceeded. In this position of the torque transfer bodies, the spring element supported on the upper torque transfer body can then be adjusted for length by applying a predefined force and the abutment is created or fixed in position in this length adjustment.

The invention thus relates to a torque-limiting device, in which a spring element is clamped between two bearing elements, whereof one forms a bearing and the other an abutment. Both the bearing and the abutment are preferably irreversibly fastened to a shaft. According to a development of the invention, it is proposed that the abutment is constituted by an intermediate element or support element. The support element can comprise a saucer-shaped section, on which one of the torque transfer bodies or the compression spring is directly supported. The support element is preferably fixed on the shaft in conjunction with a plastic deformation. This takes place in the previously described manner, in that a predefined force is applied to the support element in the axial direction of the shaft during the calibration. The teeth of the torque transfer bodies engaging into one another can stand head to head, so that the force is set which has to be overcome when the release of the limiting torque takes place. In a preferred embodiment, the support element, which can be produced from a softer material than the preferred hardened shaft, which can also form the screwdriver, has a neck, which projects from the saucer-shaped section. The inner diameter of the neck extending in the axial direction is preferably only slightly greater than the outer diameter of the shaft, so that the support element can be pushed onto the shaft. In a preferred embodiment, the end of the shaft has a recess, which in particular has a conical surface. The recess can also comprise a step. The conical surface preferably borders on an annular step. In the irreversible fixing of the support element on the shaft, material of the neck is pressed into this recess. This takes place by means of a pressing tool. The pressing takes place while the predefined force is applied to the support element in the axial direction. The material of the support element flows into the recess of the shaft in order thus to create a form-fit connection. The recess can extend around the entire shaft. In a development of the invention, provision can be made such that a sliding bearing disc is arranged between an end of the spring element and the support element. The sliding bearing disc can be formed by a washer. The contact surface of the spring element with respect to the support element is thus rotationally decoupled. A plurality of such sliding bearing discs, i.e. for example washers, can be installed. They can then be arranged in the manner of a packet above one another, so that the shaft together with the support element fastened to the shaft can rotate with respect to the spring element. The sliding bearing disc can be produced from a material typical of sliding bearings, for example steel, plastic, ceramics, graphite or from sintered materials. The selection of nonferrous alloys is also provided for. Such a sliding bearing disc can also be used in such embodiments wherein the shaft is plastically deformed in order to exert an abutment function.

### BRIEF DESCRIPTION OF THE INVENTION

Examples of embodiment of the invention are explained below with the aid of the appended drawings. The figures show the following:

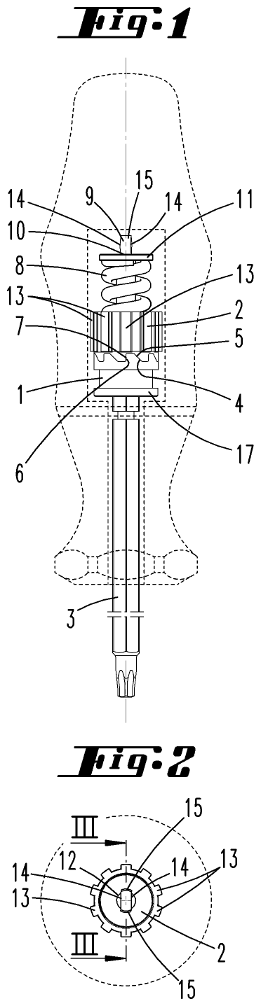

FIG. 1 a torque transfer device used in a screwdriver handle,

FIG. 2 the plan view onto the torque transfer device according to FIG. 1,

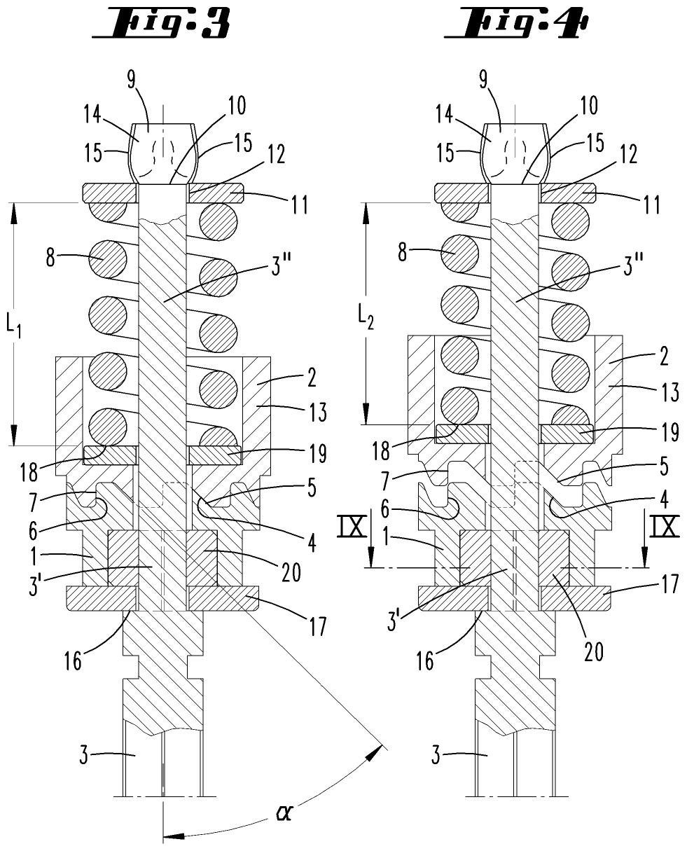

FIG. 3 the cross-section through line III-III enlarged, wherein two torque transfer bodies 1, 2 occupy their basic position and are acted upon by a pretensioned spring element 8,

FIG. 4 a representation according to FIG. 3, wherein however the two torque transfer bodies 1, 2 have been moved rotationally with respect to one another until the spring element 8 acquires a minimum length L2,

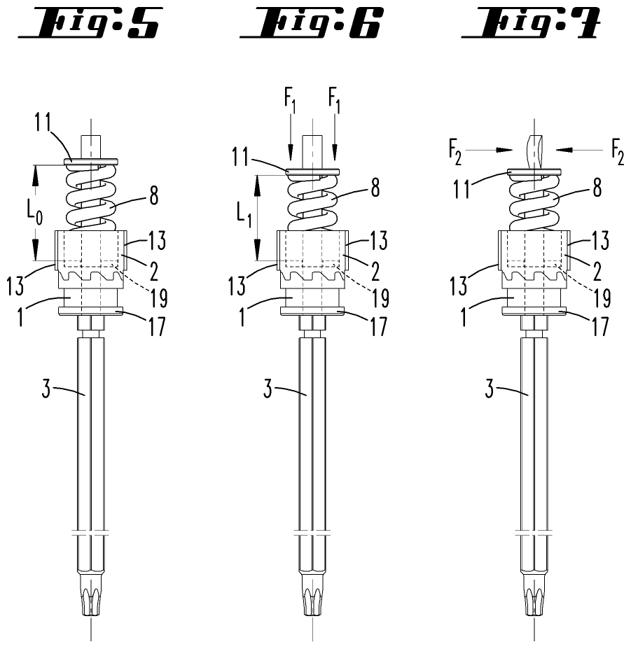

FIG. 5 a representation similar to FIG. 3, but in an assembly position, in which uncompressed spring element 8 has a neutral length L0,

FIG. 6 a representation according to FIG. 5 after a predefined force F1 has been applied to spring element 8, wherein spring element 8 is compressed to calibration length L1,

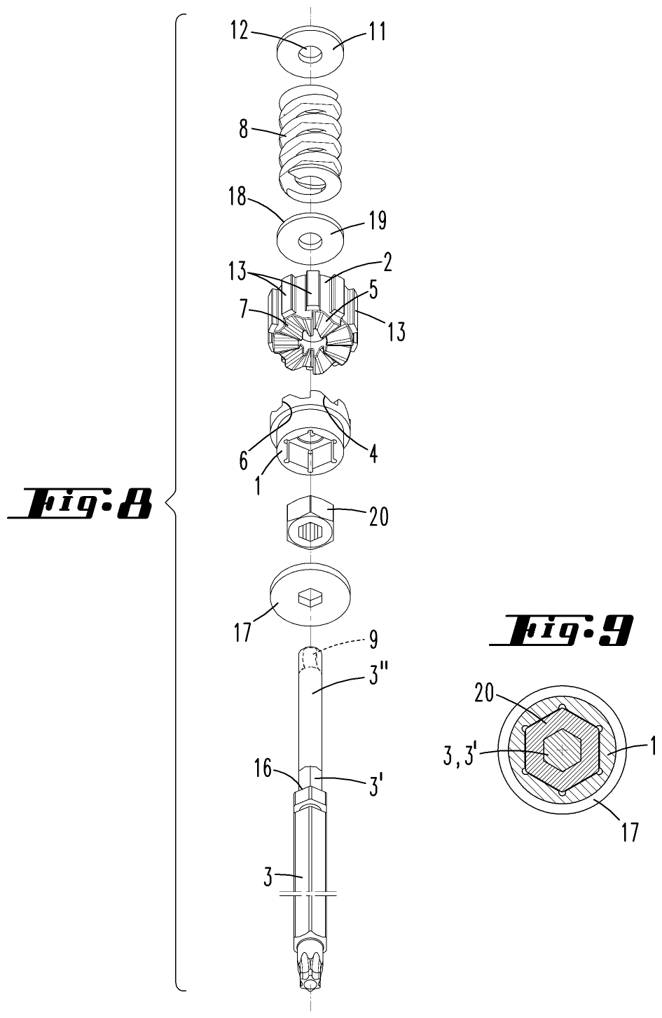

FIG. 7 a sequence representation, in which the upper end of spring element 8 is fixed in position relative to the shaft 3,

FIG. 8 an exploded representation,

FIG. 9 a cross-section through line IX-IX in FIG. 4,

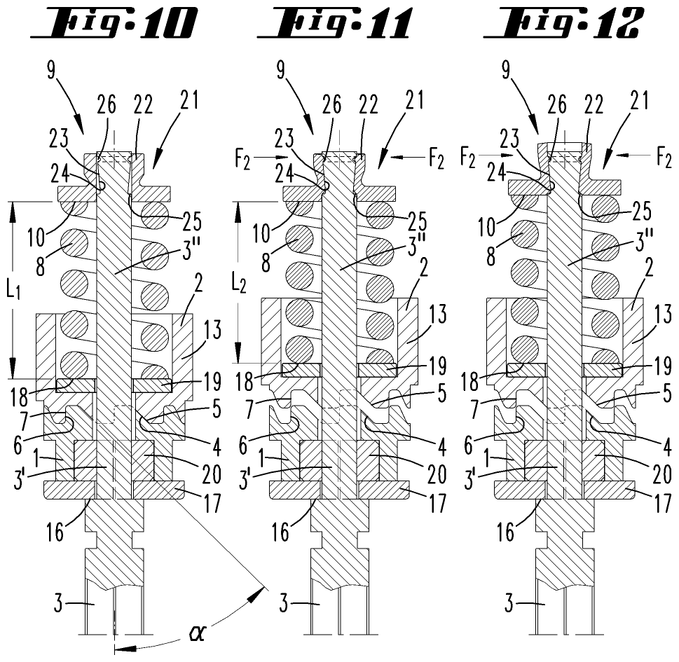

FIG. 10 a further example of embodiment of the invention in a representation according to FIG. 3,

FIG. 11 the example of embodiment represented in FIG. 10 after fixing of support element 21 to shaft 3,

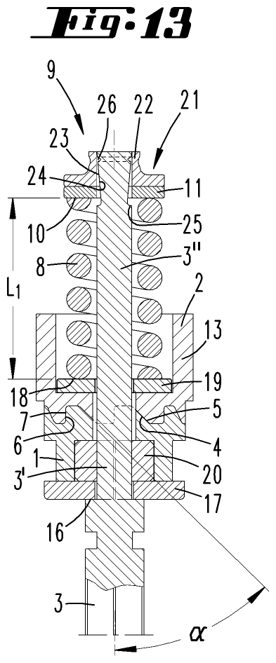

FIG. 12 a representation according to FIG. 11, but with a spring element 8 which has a different spring characteristic, wherein the support element is fastened to shaft 3 in another axial position,

FIG. 13 a representation of a further example of embodiment according to FIG. 10.

### DETAILED DESCRIPTION OF THE INVENTION

The torque transfer device comprises a shaft 3 produced from steel, which comprises a projection or a step 16, on which a first torque transfer body 1 is supported. Torque transfer body 1 produced for example as a plastic injection moulded part, a metal injection moulded part, a milled part, a precision cast part is connected rotationally fixed to shaft 3 and does not change its axial position with respect to shaft 3. First torque transfer body 1 comprises a torque transfer tooth with an oblique flank 4 and a steep flank 6 lying opposite the latter. In the example of embodiment, a washer 17 is supported on step 16, on which torque transfer body 1 is in turn supported. Torque transfer body 1 has a cavity, which is open towards washer 17, with a hexagonal inner profile, into which a hexagonal driver plate 20 produced from steel is inserted. The latter has a multi-edge inner profile, which is filled by a multi-edge outer profile section 3′ of shaft 3.

A second torque transfer body 2, which can also be constituted as a plastic injection moulded part, a metal injection moulded part, a milled part, a precision cast part or sintered part, can be moved with respect to first torque transfer body 1 in the axial direction and in the circumferential direction, relative to axis A. Second torque transfer body 2 can be connected rotationally fixed to a handle, which for example comprises a cavity, in which the torque transfer device is plugged. For this purpose, second torque transfer body 2 can comprise peripheral grooves or peripheral ribs 13 extending in the axial direction, which engage in a counter-profile of the cavity of the handle. Second torque transfer body 2 also comprises torque transfer teeth. The gaps between the two torque transfer teeth form a second oblique flank 5 and a second steep flank 7. Second oblique flank 5 lies in surface contact with first oblique flank 4. Second steep flank 7 lies in surface contact against first steep flank 6.

Second torque transfer body 2 comprises a support surface 18, on which a lower end of a spring element 8 constituted as a steel helical compression spring is supported. In the example of embodiment, support surface 18 is constituted by a metal disc 19, which lies in a cavity of torque transfer body 2.

Shaft 3 extends through spring element 8 and projects beyond the second upward-pointing end of spring element 8 and a washer forming an intermediate element 11 lying on the upper end of spring element 8, through a hole of which washer shaft 3 extends. In this state represented in FIG. 5, spring element 8, to which no force is applied, has its neutral length L0. Shaft section 3′ of shaft 3 extending through spring element 8 preferably has a round profile.

In a further assembly step, which is represented in FIG. 6, a predefined force F1 is applied in the axial direction to the upper end of spring element 8 or intermediate element 11 formed by a washer lying on said upper end. This leads to a shortening of the length of spring element 8 to a calibration length L1.

Instead of an intermediate element 11, a plurality of intermediate elements 11 can also be arranged one above the other. Intermediate elements 11 then form sliding bearing plates, which can slide against one another during a rotational movement of the shaft with respect to spring element 8.

In the following process step represented in FIG. 7, spring element 8 is fixed at this length L1. This takes place by the application of radial forces F2 on the end of shaft 3 projecting beyond the upper end of spring element 8 or the washer, which end of the shaft constitutes an abutment 9. On account of this application of force, which can take place using two pressure cheeks, the free end of shaft 3 is deformed to form abutment 9. Shoulders are thereby formed, on which the washer can be supported. Two mutually opposite wide-side surfaces 14 arise, the distance between which is smaller than the diameter of the shaft, so that a material flow towards narrow sides 15 takes place, which form the shoulders on which intermediate element 11 is supported.

The state ready for use represented in FIGS. 1 and 3 is then reached. If, in this state, first torque transfer body 1 is held fixed in position and a torque is applied in the clockwise direction to second torque transfer body 2, the two oblique flanks 4, 5 slide over one another. If a torque is applied in the opposite direction, the two steep flanks 6, 7 rest against one another.

When the torque is applied in the clockwise direction, the two oblique flanks 4, 5 slide over one another until they are in the position represented in FIG. 4. In this position, the two torque transfer bodies 1, 2 have become spaced apart from one another roughly by the height of the torque transfer tooth. Spring element 8 has been compressed from the length L1 to shortened length L2. This position corresponds to a release torque, which corresponds to the limiting torque that can be transferred by the torque-limiting device.

In a variant of the calibration of the torque-limiting device, a greater force F1 can be used in the step represented in FIG. 6, which compresses spring element 8 to length L2. The striking step of abutment 9 is then produced at a defined axial distance from the upper edge of washer 11 or spring element 8, wherein this axial distance corresponds to the maximum axial displacement path of second torque transfer body 2 with respect to first torque transfer body 1, i.e. roughly the height of the torque transfer tooth.

In a further variant of the calibration, the application of a calibration force F1 generated for example by a standard weight to spring element 8, as represented in FIG. 6, can also take place in the state represented in FIG. 4, when the calibration teeth of the two torque transfer bodies 1, 2 rest against one another.

In the further example of embodiment represented in FIGS. 10 to 12, shaft 3 is produced from a hardened material and forms the screwdriver. Provision can however also be made such that a chuck is arranged at the free end of shaft 3 for receiving a screwdriver.

It is essential that spring element 8 is supported on a saucer-shaped section of a support element 21. Essentially rotation-symmetrical support element 21 comprises a disc-shaped section, which forms support shoulders 10. Projecting from this section in the axial direction is a neck 22, the inner diameter of which is slightly larger than the outer diameter of shaft 3. This neck 22 is pushed onto the end of shaft 3 and is acted upon by an axial force in the manner described above for the calibration of the torque-limiting device. The teeth of the two torque transfer bodies 1, 2 can engage into one another, as is shown in FIG. 10. It is however also possible for the heads of the teeth of torque transfer bodies 1, 2 to rest on one another, as is shown in FIGS. 11 and 12.

The irreversible fixing of support element 21 takes place by means of a deformation force applied perpendicular to the direction of extension of the shaft. The deformation force is generated by deformation cheeks (not represented). A plurality of cheeks arranged star-shaped can be provided, which move towards one another in the radial direction in order to fasten neck 22 to the end of the shaft.

The end of the shaft comprises a recess 23, which has a conical surface 24 which is adjacent to a step 25. The preferably softer material than the material of shaft 3 of neck 22 is pressed into this recess 23.

In the example of the embodiment represented in FIG. 13, an intermediate element 11 in the form of a sliding bearing disc is arranged between the end of spring element 8 and support element 21. Sliding bearing disc 11 is constituted as a washer and can be produced from steel, plastic, ceramics, graphite, a sintered material or a nonferrous alloy. A plurality of such intermediate elements 11 can also be arranged above one another. The purpose of this intermediate element 11 is to reduce friction. When the limiting torque is exceeded, shaft 3 can rotate with respect to spring element 8. The wide-side surface of intermediate element 11 pointing towards support element 21 forms a sliding friction surface, which rotates when there is a rotation with respect to the wide-side surface of support element 21. The surface friction of wide-side surfaces lying one upon the other is reduced by a friction-reducing surface property. The invention thus also relates to a device, wherein a sliding disc 11 is arranged between spring element 8 and support element 21, which sliding disc abuts with a sliding surface against a counter-sliding surface of support element 21.

The above embodiments serve to explain the inventions covered as a whole by the application, which each also independently develop the prior art at least by the following combinations of features, wherein two, a plurality or all of these combinations of features can also be combined, namely:

A torque-limiting device, which is characterised in that abutment 9 is formed uniformly in terms of material by shaft 3 or by a support element 11, 21 fastened irreversibly to shaft 3.

A torque-limiting device, which is characterised in that support element 21 is fastened to shaft 3 thereby forming a form-fit connection in conjunction with a plastic deformation.

A torque-limiting device, which is characterised in that support element 21 forms a wide side of a saucer-shaped section that points towards spring element 8 and a neck 22, which points away from the wide side and which is connected to shaft 3 thereby forming a plastic deformation.

A torque-limiting device, which is characterised in that shaft 3 comprises a recess 23, into which material of support element 21 or a neck 22 of support element 21 is pressed.

A torque-limiting device, which is characterised in that recess 23 has a conical surface 24 and/or a step 25.

A torque-limiting device, which is characterised in that spring element 8 is supported on abutment 9 directly or with interposing an intermediate element 11, wherein provision is in particular made such that intermediate element 11 is a washer and/or that spring element 8 is supported on one of torque transfer bodies 1, 2 by interposing a second intermediate element 19, in particular a washer 19.

A torque-limiting device, which is characterised in that torque transfer bodies 1 move away from one another during the relative rotation by means of a lifting device, wherein the lifting device is formed in particular by a first oblique flank 4 of a first torque transfer body 1, which is fixedly connected to shaft 3, and a second oblique flank 5 of a second torque transfer body 2, which is rotatable with respect to shaft 3, wherein in particular provision is made such that second torque transfer body 2 is assigned rotationally fixed but axially displaceable to a handle, wherein in particular provision is made such that the torque-limiting device is arranged in a cavity of the handle, in which second torque transfer body 2 can move in the axial direction and/or second torque transfer body 2 is axially displaceable as a consequence of ribbing in a cavity of the handle.

A torque-limiting device, which is characterised by a sliding bearing disc 11 lying in sliding friction surface contact against a counter-surface of support element 11, on which sliding bearing disc spring element 8 is supported.

A method, which is characterised in that two torque transfer bodies 1, 2 are displaceable during a relative rotation about axis A in the direction of said axis A relative to one another against a restoring force of a pretensioned spring element 8, wherein spring element 8 having a neutral length L0 in the state when the force is not applied is brought from neutral length L0 to a calibrated length L1, L2 by axial application with a predefined external force F1, in which calibrated length an abutment 9 is irreversibly fixed, characterised in that length L1, L2 is fixed by a plastic deformation.

A method, which is characterised in that both a bearing 17 and abutment 9, between which spring element 8 is clamped, are fastened irreversibly to shaft 3.

A method, which is characterised in that abutment 9 is formed uniformly in terms of material by shaft 3 or by a support element 11, 21 fastened irreversibly to shaft 3.

A method, which is characterised in that the torque transfer teeth of torque transfer bodies 1, 2 rest against one another when abutment 9 is fixed.

A production batch, which is characterised in that the torque-limiting device is constituted according to any one of claims 1 to 7 and predefined force F1 is the same in the case of all the torque-limiting devices.

A production batch, which is characterised in that the torque-limiting device is formed according to a method according to any one of claims 8 to 11, wherein predefined force F1 is the same in the case of all the torque-limiting devices.

All the disclosed features are essential to the invention (in themselves, but also in combination with one another). In the disclosure of the application, the disclosure content of the associated/appended priority documents (copy of the prior application) are thus also included in their full content, also for the purpose of incorporating features of these documents in claims of the present application. The sub-claims characterize with their features, including without the features of a claim referred to, independent inventive developments of the prior art, in particular in order to make divisional applications on the basis of these claims. The invention specified in each claim can also include one or more of the features indicated in the above description, in particular provided with reference numbers and/or stated in the list of reference numbers. The invention also relates to design forms, wherein individual features stated in the above description are not implemented, in particular insofar as they can evidently be dispensed with for the given intended use or can be replaced by other means which technically have the same effect.

1-15. (canceled)

16. A torque-limiting device having two torque transfer bodies, whereof at least one is displaceable during a relative rotation about an axis in the direction of said axis with respect to the other torque transfer body against the restoring force of a spring element clamped with pretensioning between a bearing and an abutment along a shaft extending in the axis, wherein the pretensioning of the spring element is set irreversibly by the axial position of the abutment during the production of the torque-limiting device, wherein the abutment is formed uniformly in terms of material by the shaft or by a support element fastened irreversibly to the shaft.

17. The torque-limiting device according to claim 16, wherein the support element is fastened to the shaft thereby forming a form-fit connection in conjunction with a plastic deformation.

18. The torque-limiting device according to claim 16, wherein the support element forms a wide side of a saucer-shaped section that points towards the spring element and a neck, which points away from the wide side and which is connected to the shaft thereby forming a plastic deformation.

19. The torque-limiting device according to claim 17, wherein the shaft comprises a recess, into which material of the support element or of a neck of the support element is pressed.

20. The torque-limiting device according to claim 19, wherein the recess has a conical surface and/or a step.

21. The torque-limiting device according to claim 16, wherein the spring element is supported on the abutment directly or by interposing an intermediate element, and/or that the spring element is supported on a washer and/or that the spring element is supported on a torque transfer body by interposing a second intermediate element, in particular a washer.

22. The torque-limiting device according to claim 16, comprising: torque transfer bodies which move away from one another during relative rotation by means of a lifting device, wherein the lifting device is formed by a first oblique flank of a first torque transfer body which is fixedly connected to a shaft, and a second oblique flank of a second torque transfer body which is rotatable with respect to the shaft, or that a second torque transfer body is assigned rotationally fixed but axially displaceable to a handle, or that the torque-limiting device is arranged in a cavity of the handle, in which a second torque transfer body can move in the axial direction or that a second torque transfer body is axially displaceable as a consequence of ribbing in a cavity of the handle.

23. The torque-limiting device according to claim 16, wherein a sliding bearing disc lying in sliding friction surface contact against a counter-surface of the support element, on which sliding bearing disc the spring element is supported.

24. The torque-limiting device according to claim 16, wherein the bearing is fastened irreversibly to the shaft.

25. The torque-limiting device according to claim 16, wherein the support element fixes in position the length of the spring element calibrated by application of a predefined force to the spring element.

26. A method for calibrating a torque-limiting device, with two torque transfer bodies, whereof at least one is displaceable during a relative rotation about an axis in the direction of said axis with respect to the other torque transfer body against the restoring force of a spring element clamped with pretensioning between a bearing and an abutment along a shaft extending in the axis, wherein the pretensioning of the spring element is set irreversibly by the axial position of the abutment during the production of the torque-limiting device, wherein the abutment is formed uniformly in terms of material by the shaft or by a support element fastened irreversibly to the shaft, the method including the spring element having a neutral length in the state when the force is not applied is brought from the neutral length to a calibrated length by axial application with a predefined external force, in which calibrated length an abutment is irreversibly fixed, and wherein the length is fixed by a plastic deformation.

27. The method according to claim 26, wherein both a bearing and the abutment, between which the spring element is clamped, are irreversibly fastened to a shaft.

28. The method according to claim 26, wherein the abutment is formed uniformly in terms of material by the shaft or by a support element fastened irreversibly to the shaft.

29. The method according to claim 26, wherein the torque transfer teeth of the torque transfer bodies rest against one another when the abutment is fixed.

30. A production batch of torque transfer tools which comprises a plurality of screwdrivers with in each case a torque-limiting device, wherein the torque-limiting device is constituted having two torque transfer bodies, whereof at least one is displaceable during a relative rotation about an axis in the direction of said axis with respect to the other torque transfer body against the restoring force of a spring element clamped with pretensioning between a bearing and an abutment along a shaft extending in the axis, wherein the pretensioning of the spring element is set irreversibly by the axial position of the abutment during the production of the torque-limiting device, wherein the abutment is formed uniformly in terms of material by the shaft or by a support element fastened irreversibly to the shaft and the predefined force is the same in the case of all the torque-limiting devices.

31. A production batch of torque transfer tools which comprises a plurality of screwdrivers with in each case a torque-limiting device having two torque transfer bodies produced according to a method wherein at least one of the two torque transfer bodies is displaceable during a relative rotation about an axis in the direction of said axis with respect to the other torque transfer body against the restoring force of a spring element clamped with pretensioning between a bearing and an abutment along a shaft extending in the axis, wherein the pretensioning of the spring element is set irreversibly by the axial position of the abutment during the production of the torque-limiting device, wherein the abutment is formed uniformly in terms of material by the shaft or by a support element fastened irreversibly to the shaft, wherein the spring element having a neutral length in the state when the force is not applied is brought from the neutral length to a calibrated length by axial application with a predefined external force, in which calibrated length an abutment is irreversibly fixed, wherein the length is fixed by a plastic deformation, and wherein the predefined force is the same in the case of all the torque-limiting devices.
